(19) United States
(12) Patent Application Publication
(10) Pub. No.: US 2008/0093211 A1
(43) Pub. Date: Apr. 24, 2008

(54) Method for site-selective functionalization of carbon nanotubes and uses thereof

(75) Inventors: Ganapathiraman Ramanath, Clifton Park, NY (US); Raghuveer S. Makala, Clifton Park, NY (US)

Correspondence Address: FOLEY AND LARDNER LLP;SUITE 500, 3000 K STREET NW, WASHINGTON, DC, 20007, US

(73) Assignee: Rensselaer Polytechnic Institute

(21) Appl. No.: 11/645,008

(22) Filed: Dec. 26, 2006

Related U.S. Application Data
(60) Provisional application No. 60/754,058, filed on Dec. 27, 2005.

(51) Int. Cl. B01J 19/08; C07C 229/22; C07C 69/00

(52) U.S. Cl. 204/157.63; 560/1; 560/169; 977/746

(57) ABSTRACT

A method of functionalizing a carbon nanotube includes providing a carbon nanotube, irradiating at least one exposed portion of the nanotube surface with ions to generate defect sites on the at least one exposed portion, and forming at least one functional group at a defect site. The method optionally includes attaching a nanostructure to the at least one functional group.

### CROSS-REFERENCE TO RELATED PATENT APPLICATIONS

This application claims priority to U.S. Provisional Application 60/754,058, filed on Dec. 27, 2005, which is incorporated herein by reference in its entirety.

### STATEMENT REGARDING FEDERALLY-SPONSORED RESEARCH OR DEVELOPMENT

This invention was made with Government support under DMR 9984478 awarded by the National Science Foundation. The Government has certain rights in the invention.

### BACKGROUND OF THE INVENTION

The present invention relates generally to the field of carbon nanotubes and specifically to the site-selective functionalization of carbon nanotubes.

Carbon nanotubes (CNTs) have been functionalized by several different methods, including acid-based wet-chemical oxidation, amidation, estrification, diimide-activation and solubilization, and hydrophobic adsorption of aromatic derivatives. These strategies typically rely on random defect creation or adsorption, which do not allow precise control over the location of the functional group on the CNT surface.

Functionalized CNTs have many potential applications due to their mechanical, electrical and electronic properties. However, the difficulty in controlling the location and type of functionalization hinders some of these applications.

### SUMMARY OF THE INVENTION

One embodiment of the invention relates to a carbon nanotube comprising at least one functional group in a site-selective functionalization on the surface of the nanotube or a plurality of functional groups in an ordered arrangement on the surface of the nanotube.

Another embodiment of the invention relates to a method of functionalizing a carbon nanotube comprising providing a carbon nanotube, providing ions at a dose greater than 10¹³ ions cm⁻² having an energy greater than 1 keV on at least one first portion of the nanotube surface, and exposing the nanotube to an oxygen-containing medium such that at least one functional group is formed on the at least one first portion of the nanotube surface.

Another embodiment of the invention relates to a method of functionalizing a carbon nanotube, comprising providing a carbon nanotube, irradiating at least one exposed portion of the nanotube surface with ions to generate defect sites on the at least one exposed portion, and forming at least one functional group at a defect site.

### BRIEF DESCRIPTION OF THE DRAWINGS

FIGS. 1A, 1B, 5A, 6A, 6B, 7A, and 8A are schematic illustrations of CNTs according to the embodiments of the present invention. FIG. 1A shows ordered and site-selective functionalization of a carbon nanotube. FIG. 1B shows a device that is adapted to detect the selective attachment of a nanostructure. FIG. 5A shows attachment of Au nanoparticles onto a CNT. FIG. 6A shows attachment of fluorescent nanospheres. FIG. 6B shows attachment of fluorescent nanospheres onto a drop-coated mat of CNTs. FIG. 7A shows attachment of lysine containing Au nanoparticle markers onto a CNT. FIG. 8A shows attachment of azurin containing Au nanoparticle markers onto a CNT.

FIGS. 2A and 8D are plots of measured micro-Raman intensity versus wavenumber of CNTs according to the embodiments of the present invention. FIG. 2A shows micro-Raman spectra for both non-irradiated and irradiated CNTs. FIG. 8D shows micro-Raman spectra for pristine azurin, and for non-irradiated CNTs and irradiated CNTs after treatment with azurin.

FIG. 2B is a plot of measured Fourier transform infrared (FTIR) absorbance versus wavenumber of CNTs according to the embodiments of the present invention. FIG. 2B shows FTIR spectra for both non-irradiated and irradiated CNTs after exposure to air.

FIGS. 3A, 3B, 4A, 4B, and 5D are transmission electron microscopy (TEM) images of carbon nanotubes according to the embodiments of the present invention. FIG. 3A shows CNTs after irradiation by Ga⁺ ions (10¹⁶ cm⁻², 10 keV), wherein the white dotted lines represent the ion beam track. FIG. 3B shows a magnified view of the circled region in FIG. 3A in which lattice fringes from the graphene cylinders are visible. The images demonstrate that the multiwalled CNT structure is preserved even for CNTs with diameters about 20 nm. FIGS. 4A and 4B show the graphitic basal planes of non-irradiated and irradiated (Ga⁺ ions, 10¹⁶ cm⁻², 10 keV) CNTs, respectively. The images demonstrate that the crystalline structure is preserved upon irradiation under these conditions, and the large number of discontinuities and increased curvature of the basal planes suggest the generation of point defects during irradiation. FIG. 5D is a scanning TEM (STEM) image of site-selective attachment of Au nanoparticles on CNT bundles. The ion-irradiated portion of the underlying SiN membrane is damaged and sputtered away. The diffraction rings show that the Au nanoparticles attached to the irradiated portion of the CNT bundles possess a face-centered cubic (FCC) structure.

FIGS. 5B, 5C, 6D, 7B, 7C, 8B, and 8C are scanning electron microscopy (SEM) images of carbon nanotubes according to the embodiments of the present invention. FIG. 5B shows attachment of Au nanoparticles on an aligned CNT bundle. The 500-nm dark band corresponds to the path traversed by a 10¹⁷ cm⁻² 10 keV Ga⁺ ion beam (middle image in FIG. 5B). Bright spots on irradiated portions of CNTs (top image in FIG. 5B) are Au nanoparticles, which are not observed on non-irradiated portions of CNTs (bottom image in FIG. 5B). FIG. 5C shows attachment of Au nanoparticles on a drop-coated mat of CNTs irradiated by Ar⁺ ions (10¹⁶ cm⁻², 5 keV), wherein the same region of the CNT mat was imaged under secondary electron imaging (top image in FIG. 5C) and atomic-number contrast imaging (bottom image in FIG. 5C). FIG. 6D shows attachment of fluorescent nanospheres on a drop-coated mat of CNTs following Ar⁺ irradiation (5×10¹⁷ cm⁻², 5 keV). FIGS. 7B and 7C show attachment of Au-labeled lysine molecules on irradiated and non-irradiated portions of dispersed CNTs, respectively. FIGS. 8B and 8C show attachment of Au-labeled azurin proteins on irradiated and non-irradiated portions of dispersed CNTs, respectively.

FIG. 5E is a plot of measured energy dispersive X-ray spectroscopy (EDX) intensity versus energy of carbon nanotubes according to the embodiments of the present invention. FIG. 5E shows EDX spectra for both non-irradiated and irradiated CNTs after immersion in a solution containing Au nanoparticles. The peak corresponding to Au Mα on the irradiated CNT, but not on the non-irradiated CNTs, indicates site-selective attachment of Au nanoparticles.

FIG. 6C is a fluorescent micrograph of carbon nanotubes according to the embodiments of the present invention. FIG. 6C shows attachment of fluorescent nanospheres on a 1 mm×5 mm “†”-shaped macropattern created by Ar⁺ irradiation (5×10¹⁷ cm⁻², 5 keV).

FIG. 7D is a plot of measured x-ray photoelectron spectroscopy (XPS) intensity versus binding energy of carbon nanotubes according to the embodiments of the present invention. FIG. 7D shows XPS spectra of C 1 s, N 1 s, and Au 4 f core levels obtained from lysine (Au labeled) attached onto irradiated CNTs via amide bond formation.

### DETAILED DESCRIPTION OF THE PREFERRED EMBODIMENTS

In a first preferred embodiment, the present inventors have discovered that CNTs may be functionalized at predetermined locations on the CNT surface. At least one functional group (e.g., a single atom or a single group of atoms) is formed on the CNT surface in a site-selective functionalization of the CNT surface, preferably on ion induced defects sites on the CNT surface. A plurality of functional groups are arranged in an ordered arrangement on at least one portion of the CNT surface. Of course, if desired, a plurality of functional groups may be formed on more than one portion of the CNT surface, either in a serial fashion or simultaneously, and may, but need not, cover substantially the entire CNT surface. Of course, if desired, more than one type of functional group (e.g., carboxyls and allyls) may be formed on different portions of the CNT surface.

Preferably but not necessarily, focused ion beam (FIB) irradiation is used to functionalize multiwalled CNTs at predetermined locations. This approach involves the use of ions having an energy of at least 1 keV to irradiate particular locations of the CNT surface, such as particular segments of CNT axis, so as to site-selectively form at least one functional group on those particular locations. An arrangement of functional groups is ordered so long as its location on the CNT surface is not random. The concentration of functional groups on the irradiated portions of the CNT surface is higher than on the non-irradiated portions. For instance, the non-irradiated portions contain substantially no functional groups when measured microscopically or spectroscopically. The size of the irradiated locations, and hence the size of the functionalized portions of the CNT surface, can be adjusted down to a few nanometers by using increasingly smaller FIB spot sizes. Additional or alternative methods can be employed. For instance, irradiating through lithographic masks, or irradiating with scanning probe-tips or related near-field modification methods, can decrease the functionalized portions of the CNT surface down to atomic levels. Different types of ions may be used. For instance, Ga⁺ or Ar⁺ ions yield similar results, indicating that functionalization is independent of the projectile species used.

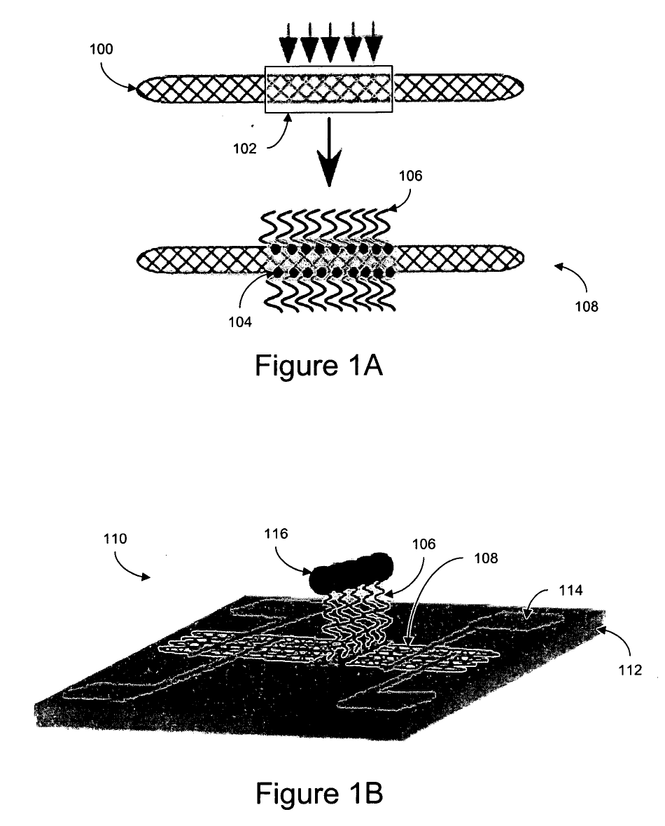

FIG. 1A shows exemplary formation of ordered and site-selective functional groups on precise locations of the CNT surface. A carbon nanotube 100, such as a single-walled carbon nanotube (SWNT) or multiwalled carbon nanotube (MWNT), is irradiated with energetic ions, such as Ga⁺ or Ar⁺ ions, on a predefined location 102 of the nanotube surface. The location 102 may be defined by raster-scanning a focused ion beam within a defined area of the sample surface with micro- or nano-scale spatial resolution. Without wishing to be bound to any particular theory, the present inventors believe that ion irradiation of CNTs generates defects 104, such as vacancy clusters or unsaturated bonds, on the irradiated portion of the nanotube surface. These defects 104 are reactive sites that facilitate the binding of functional groups 106 selectively at those defect sites on the irradiated portions of the nanotube surface. For instance, a covalent bond is formed between the functional groups 106 and the defects 104, such as a covalent bond formed when an oxygen atom saturates an unsaturated CNT bond. Alternatively, a Van der Waals bond is formed between the functional groups 106 and the defects 104, such as a Van der Waals bond formed between a functional group and a defect site on the nanotube surface having an electronic density that is different from that of other portions of the nanotube surface. The arrangement of the functional groups 106 is ordered and not random because the functional groups 106 appear localized to a particular portion of the surface of the nanotube 100. For instance, the concentration of functional groups 106 is greater in the middle portion of the nanotube surface than on other portions. Types of functional groups include, but are not limited to, an alcohol, a carbonyl, a carboxyl, and an allyl. Oxygen-containing functional groups, such as alcohols, carbonyls, and carboxyls, may be formed by exposing the defects 104 to an oxygen-containing medium, such as air. Other types of functional groups may be formed by exposing the defects 104 to other types of controlled chemical ambients, such as hydrogen or halogen ambients. Functional groups containing carbon-carbon double- or triple-bonds, such as allyls, may, but need not, form spontaneously from the defect 104 even in the absence of a chemical ambient.

FIG. 1B shows an example of a device 110 comprising the functionalized carbon nanotube 108 of FIG. 1A. The nanotube 108 is disposed on a substrate 112 with electrical contacts 114 contacting the nanotube and disposed on opposite sides of the functional groups 106 located on the surface of the nanotube 108. The functional groups 106 are located on the nanotube surface opposite the substrate 112, for example the functional groups 106 are located on the entire exposed circumference of the nanotube, such that the functional groups 106 are readily accessible for site-selective attachment with nanostructures 116 that are deposited onto the device 110 from an aqueous solution. Preferably, but not necessarily, the attachment is chemically specific such that a given functional group or nanostructure is capable of binding only with one type of nanostructure. For instance, the attachment comprises protein-analyte interactions, such as streptavidin-biotin interactions wherein streptavidin is a first nanostructure known to specifically bind with biotin, a second nanostructure. The device 110 is adapted to detect an attachment of at least one of (1) a nanostructure 116 with a functional group 106, or (2) a nanostructure 116 with a second nanostructure. Preferably but not necessarily, the device 110 is adapted to detect the attachment of a nanostructure by monitoring for a change in the electrical conductivity between the contacts 114. For instance, an attachment involving a reduction/oxidation (redox) reaction between the nanostructure and the functional group results in the addition or removal of at least one electron to or from the functional group, which may be detected as a change in the nanotube's conductivity. Alternatively, the presence of an attached nanostructure may induce an electric field in the carbon nanotube and alter its conductivity, such as in a CNT-based chemical field effect transistor (ChemFET). Alternatively, detection may be performed by optical means. Of course, a nanotube with plural portions of its surface functionalized with plural types of functional groups can be used to simultaneously detect for the presence of plural types of nanostructures. Optionally, plural devices 110 may comprise an array of devices that detects attachment of plural types of nanostructures, wherein each device 110 detects for the presence of a certain type of nanostructure. For instance, the array provides detection, analysis, and separation of biomolecules on a single chip. The separation may be achieved using conventional microfluidic separation devices.

The CNTs may comprise single-walled or multiwalled carbon nanotubes, and my be prepared by a variety of methods, such as by chemical vapor deposition (CVD) or by the arc discharge method. The CNTs may comprise dispersed or aligned bundles. In one aspect of the invention, dispersed CNT bundles comprise a dense mat of CNTs, drop-coated from a toluene solution and air-dried on a silicon substrate. In another aspect of the invention, aligned CNT bundles are formed by selective CVD growth on silicon oxide templates, such as on lithographically patterned silicon oxide templates, as described in United States published application US-2003-0165418-A1, incorporated herein by reference in its entirety.

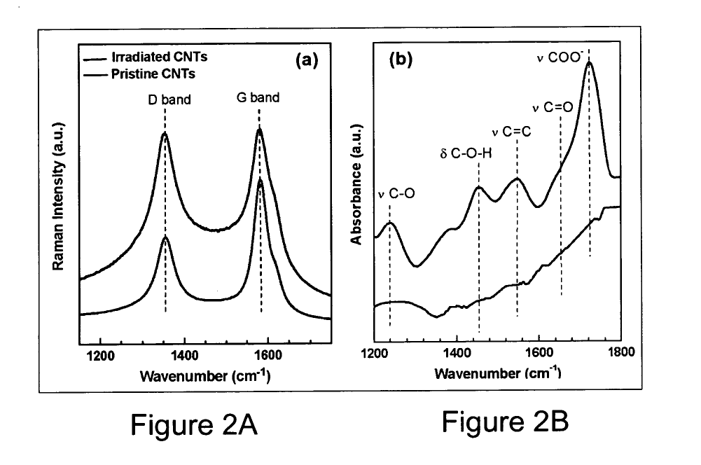

FIGS. 2A and 2B show selective defect creation and functionalization of irradiated portions of CNTs. FIG. 2A shows micro-Raman spectra (spot size ˜1 μm) for both non-irradiated and irradiated CNTs, wherein a larger D band for irradiated CNTs as compared to non-irradiated CNTs indicates a higher defect concentration for irradiated CNTs. FIG. 2B shows the FTIR spectra of CNTs irradiated with Ar⁺ ions (10¹⁷ cm⁻², 5 keV) and subsequently exposed to air. The FTIR spectra for irradiated CNTs show absorbance intensities at 1723, 1650, 1547, and 1455 cm⁻¹, which correspond to the chemical signatures of the functional groups O═C—O, C═O, C═C, and C—O—H, respectively. Spectra from non-irradiated CNTs do not show any detectable amounts of these functional groups. Without wishing to be bound by any particular theory, the present inventors believe that the high momentum transfer cross-sections (e.g., ˜5×10⁻⁶ nm² for 30 keV Ga⁺ ions) and the high energy density (˜420 eV/nm) imparted by ions having an energy of at least 1 keV leads to the formation of defects, such as vacancy clusters or unsaturated bonds, on the irradiated portions of the CNT surface. These defects, it is believed, are sites of increased chemically reactive which enable site-selective functionalization of the irradiated portions of the CNT surface by reaction with water and oxygen during air-exposure. The ability to localize the defects to particular segments of the CNT surface allows for spatially resolved functionalization of CNT segments. Exposure to other types of chemical ambients besides air allows for the formation of other types of functional groups.

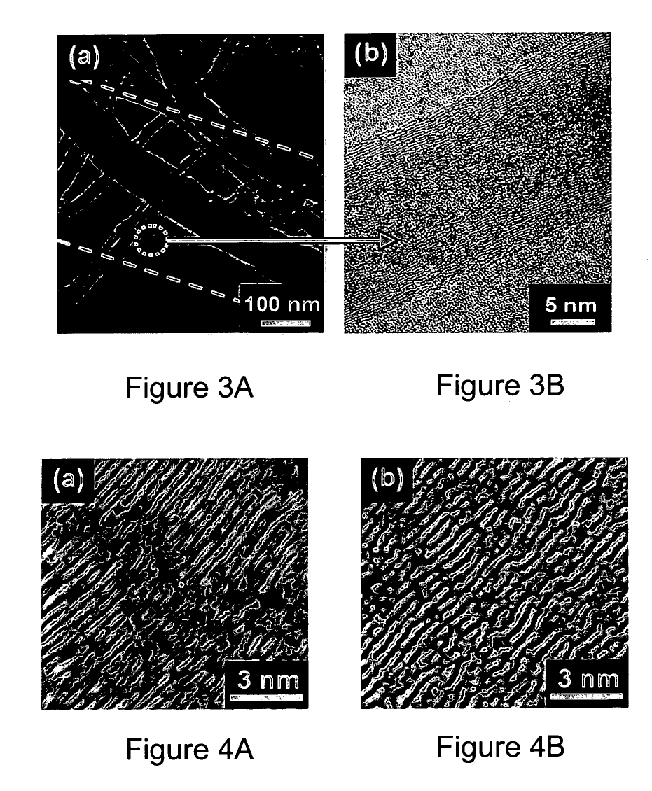

To further probe the nature of the CNT defect structure, the present inventors irradiated CNTs using Ga⁺ ions (10¹⁶ cm⁻², 10 keV) and imaged the CNTs under TEM. FIG. 3A shows a dispersed CNT bundle, wherein the white dotted lines encompass the ion beam track. FIG. 3B is a TEM micrograph of an ion-irradiated portion of a CNT showing lattice fringes from the graphene cylinders. The cylindrical hollow of the irradiated CNTs are clearly seen in both images. These images confirm that the tubular CNT structure is preserved even for ˜20-nm-diameter CNTs. FIGS. 4A and 4B are high resolution TEM micrographs showing the graphitic basal planes of non-irradiated and irradiated (10¹⁶ cm⁻², 10 keV Ga⁺ ions) CNTs, respectively. The larger number of discontinuities and increased curvature of the basal planes in the irradiated CNT of FIG. 4B suggests the generation of point defects during irradiation. These images confirm that the crystalline structure of CNTs is preserved upon irradiation with 10 keV Ga⁺ ions of a dose less than 10¹⁸ cm⁻², preferably with a dose of about 10¹⁶ cm⁻² or less. These local site defects probably, but not necessarily, alter the electrical properties of CNTs and should be accounted for when irradiated CNTs are used for device applications.

In one preferred embodiment of the present invention, the functional group provides site-selective attachment of nanostructures to the CNTs. The attachment may comprise electrostatic or covalent attachment. The attachment may comprise an intermediary attachment entity, such as a polyelectrolyte that electrostatically binds between the nanostructure and the functional group of the CNT surface. The attachment may be performed by any suitable attachment chemistry, such as by a displacement reaction between allyl bromide and a carboxylic acid. Nanostructures include, but are not limited to, nanoparticles, such as metal nanoparticles, nanospheres, amino acids, and proteins. A nanostructure may be greater than 1,000 nanometers but is generally not visible to the naked eye. For instance, a nanostructure may be a microsphere, such as a Nile Red microsphere (Molecular Probes F-8784).

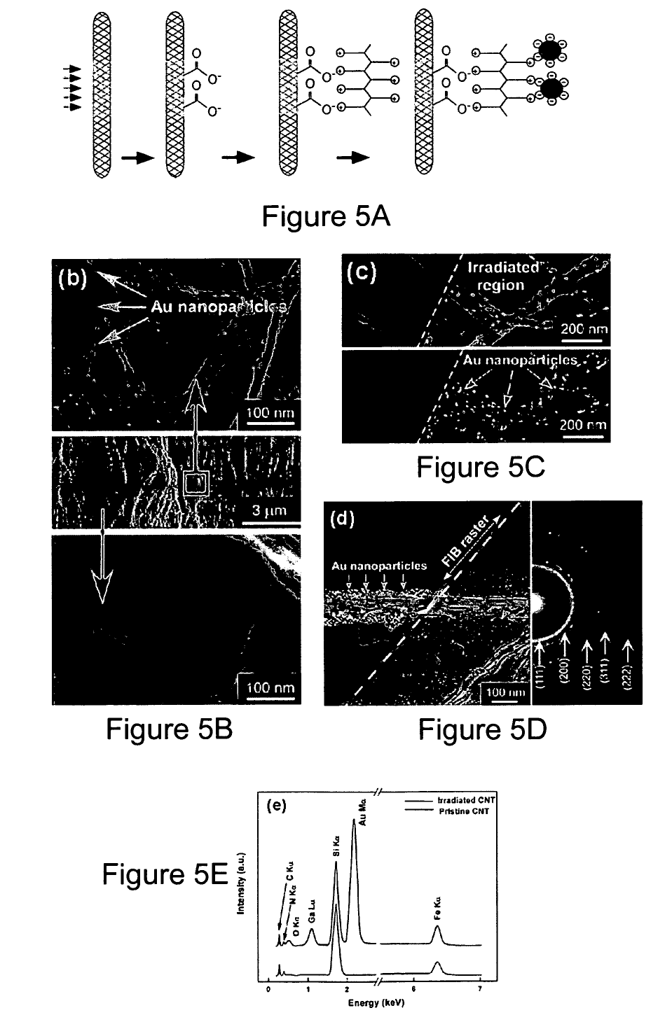

FIGS. 5A-D demonstrate site-selective attachment of a gold nanoparticle to a functionalized CNT by electrostatic interactions. In one embodiment of the invention, the nanostructure comprises a gold nanoparticle, such as a negatively-charged gold nanoparticle, that is selectively attached to at least one functional group, such as carboxyl group, via a cationic polyelectrolyte. Multiwalled CNTs were synthesized either by CVD or by the arc discharge method. FIB irradiation experiments were carried out on aligned or dispersed multiwalled CNT bundles, in a FEI Strata DB-235 dual-beam system. Irradiation by Ar⁺ ions was carried out on drop-coated CNT films in an ultra-high vacuum chamber fitted with a Perkin-Elmer model 04-303 differential ion gun. For the FIB experiments, aligned CNT bundles grown selectively on lithographically patterned templates of silica were used. 50 nm beams of focused ions (10-30 keV) were rastered across 300 to 800 nm-wide segments of aligned CNT arrays. Although smaller segments (e.g., 5 nm, determined by the focused ion beam spot size) can be functionalized, the present inventors deliberately chose larger length scales in order to allow facile visualization of the functionalized areas using conventional electron microscopy and spectroscopy techniques. The CNT arrays that were rastered by FIB irradiation were air-exposed and treated with poly(diallyldimethylammonium)-chloride (PDADMAC), a cationic polyelectrolyte known to enable electrostatic immobilization of gold nanoparticles on carboxylated CNTs. See K. Jiang et al., Nano. Lett., 3, 275 (2003). FIG. 5A shows the nanoparticle attachment scheme. The irradiated sample was immersed in an aqueous solution of PDADMAC, MW ˜100,000-200,000 and 1 mM NaCl solution for 30 min. The sample was thoroughly rinsed with a 1 mM NaCl solution and deionized water to remove loosely adsorbed polyelectrolytes, and immersed in a solution containing negatively charged gold nanoparticles (˜110 nm diameter) for 15 min followed by thorough washing with deionized water. For TEM and STEM examination, the irradiation and attachment experiments were carried out on drop-coated films of CNTs formed on 30-nm-thick electron-transparent SiN membrane windows by solvent evaporation. FIG. 5B shows that the gold nanoparticles attach only to the irradiated segments of the CNT, indicating an ordered arrangement and a site-selective functionalization of the CNTs with carboxyl groups. The 500-nm dark band corresponds to the path traversed by a 10¹⁷ cm⁻² 10 keV Ga⁺ ion beam. Bright spots on irradiated CNT segments (top image in FIG. 5B) are gold nanoparticles, which were not observed in unirradiated CNT segments (bottom image in FIG. 5B). No observable attachment is detected in the unirradiated regions. The nanoparticles are not dislodged from the irradiated segments despite repeated washing and rinsing, indicating strong electrostatic anchoring. Typically, nanoparticle anchoring is observed when CNTs are irradiated with 10¹⁵-10¹⁷ cm² of Ga⁺ ions at 10-30 keV, whereas dosing CNTs with ≦10¹³ ions cm² does not result in any attachment. Some of the irradiated CNTs in the top image of FIG. 5B are welded together due to ion irradiation. This welding is suppressed by lowering the ion dosage below 10¹⁷ cm⁻², for instance doses of about 10¹⁶ cm⁻² or less for Ga⁺ ions. FIG. 5C shows that CNTs irradiated with 5 keV Ar⁺ ions yield similar results, suggesting that the projectile species does not have a significant effect on defect creation and nanoparticle anchoring characteristics in this ion energy window for Ar⁺ ions. FIG. 5D is a STEM image of site-selective attachment of gold nanoparticles on aligned CNT bundles. The irradiated portion of the underlying SiN membrane is damaged and sputtered away, and gold nanoparticles are seen only on the irradiated portion of the CNT. The diffraction rings in FIG. 5D show that the Au nanoparticles attached to the irradiated portion of the CNT possess an FCC structure. FIG. 5E is an EDX spectra which reveals a peak corresponding to Au Mα on the irradiated CNT, but not on the non-irradiated CNTs, indicates site-selective attachment of gold nanoparticles to functionalized CNTs.

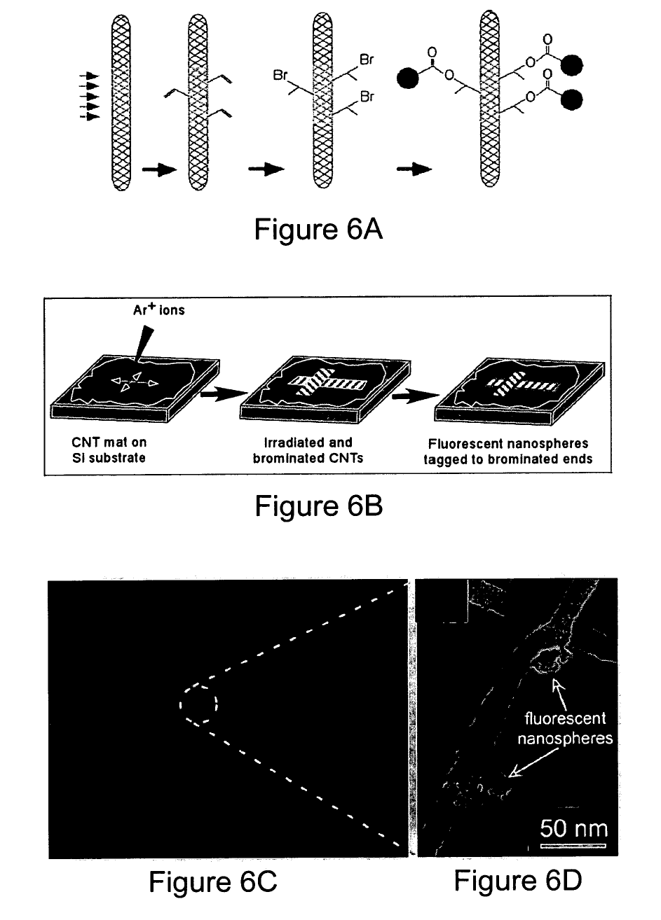

FIGS. 6A-D demonstrate site-selective attachment of a nanosphere to a functionalized CNT by covalent interactions. In one embodiment of the invention, the nanosphere comprises a carboxylated Nile-red fluorescent nanosphere, which displaces the bromine of a brominated allyl group in order to covalently bind to at least one allyl on the CNT surface. FIG. 6A shows the nanosphere attachment scheme. FIG. 6B shows the experimental setup. A mat of CNTs was drop-coated from a toluene solution and air-dried on a Si substrate. Allyl groups are generated on the CNT surface during ion irradiation with 5×10¹⁷ cm⁻² of 5 keV Ar⁺ ions to a form a “†”-shaped macropattern (1 mm×5 mm for facile optical observation). The sample was treated with a few drops of HBr in CCl₄, placed in a 0.1 M NaOH bath for 5 sec to neutralize the acid, thoroughly washed with deionized water, and dried in air. The sample was further treated with a 50 mM MES buffer solution, and 500 μl of a 2% aqueous suspension of Nile Red microspheres (Molecular Probes F-8784) was added to the covered bath. The bath was swirled for a few seconds, removed and air-dried for fluorescence microscopy imaging with a green filter. All procedures involving the fluorescent microspheres were performed in the dark prior to imaging. The carboxylated nanospheres displace bromine, revealing the regions where allyl groups are present. FIG. 6C is a fluorescence microscopy image under a green filter showing that only the regions with irradiated CNTs are selectively decorated with the nanospheres. Without wishing to be bound by any particular theory, the present inventors believe that the greater fluorescence intensity near the periphery of the irradiated macropattern suggests that the CNTs in these regions have the highest defect concentration. The relatively lower intensity near the center of the macropattern may be due to the sputtering of the CNTs due to high ion dose resulting from overlapping passes of the ion beam during rastering. FIG. 6D confirms that the fluorescent nanospheres preferentially agglomerate at defects sites, such as bent portions of the CNTs known to arise through the formation of nonhexagonal ring pairs. See A. Kumar et al., Langmuir, 16, 9775 (2000).

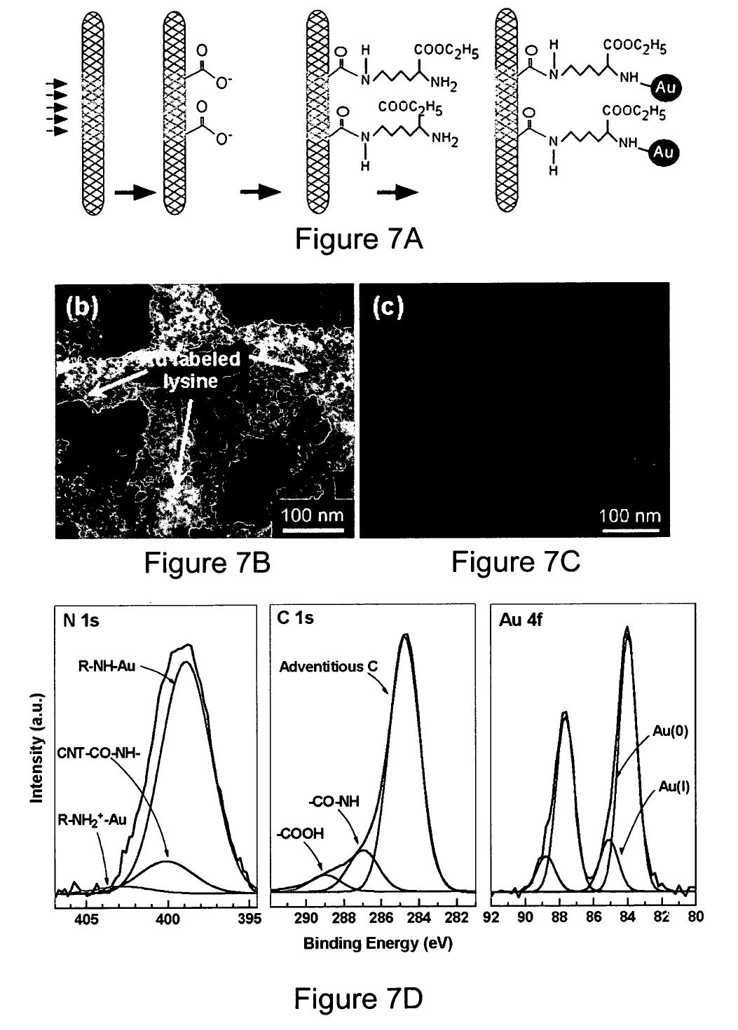

FIGS. 7A-D demonstrate site-selective attachment of an amino acid to a functionalized CNT. In one embodiment of the invention, the amino acid comprises a lycine molecule bound to irradiated CNTs via the amide bond of a carboxyl group.

FIG. 7A shows the lysine attachment scheme. A mat of CNTs was irradiated by Ar⁺ ions (10¹⁶ ions cm⁻², 5 keV) and immersed in 15 ml deionized water containing 155 mg of the amide-forming mediator 1-ethyl-3-(3-dimethyl-amino-propyl)carbodiimide (EDAC). Next, 350 mg of L-lysine ethyl ester dihydrochloride 95% was added after 2 hours and the entire solution was left undisturbed for 24 hours. The samples were subsequently washed thoroughly with deionized water to remove loosely adsorbed reaction products and immersed in an Au nanoparticle hydrosol (pH ˜8.5) for 3 hours. The Au nanoparticle markers bind strongly to amino acids for visualization of selective attachment by SEM. The samples were again rinsed thoroughly with deionized water and dried in air prior to characterization by SEM and XPS. FIGS. 7B and 7C show attachment of Au-labeled lysine molecules on irradiated CNTs but not on non-irradiated CNTs, respectively, thus confirming site-selective attachment. Additionally, EDX analysis shows N—K_{α}, O—K_{α}, and Au M_{α} X-ray peaks in EDX spectra only in irradiated CNTs, further confirming selective anchoring of lysine to irradiated CNTs. FIG. 7D shows XPS measurements that indicate that lysine is anchored to carboxyl groups in irradiated CNTs via amide bonds. This is seen from two characteristic amide signatures observed in lysine-derivatized CNTs: a N 1 s sub-band centered at 400.1 eV and a C 1 s sub-band centered at 286.9 eV. The sub-bands at 398.9 and 402.8 eV arise from the un-ionized and ionized amine groups bound to the Au nanoparticles surface, and are consistent with the presence of a higher Au 4 f_{7/2} sub-band corresponding to Au(I) seen at 85.1 eV spectra in addition to the Au(0) state at 84 eV. The absence of imide and amine sub-bands at 399.1 and 400.3 eV, respectively, seen in the intermediate complex of CNTs and EDAC indicates that lysine displaces substantially all EDAC.

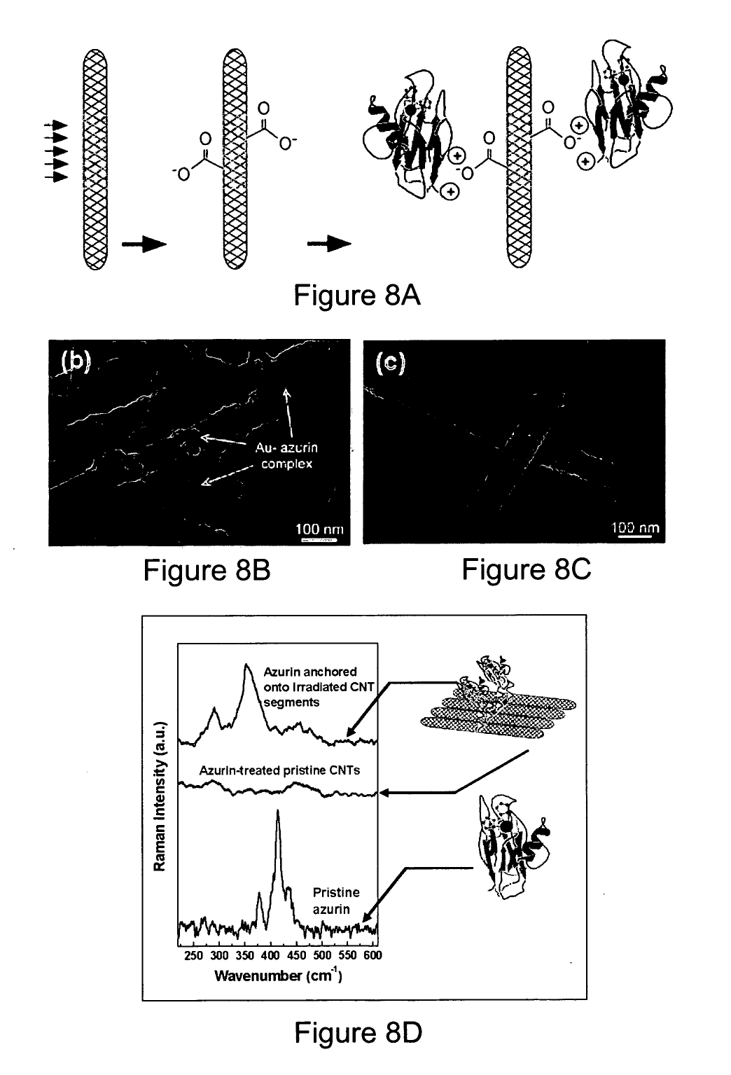

FIGS. 8A-D demonstrate site-selective attachment of a protein to a functionalized CNT. In one embodiment of the invention, the protein comprises azurin (Pseudomonas Aeruginosa), a metalloprotein with tunable electron transfer properties and a potential cancer-fighting agent, which is bound to the CNT via electrostatic interaction with a carboxyl group on the CNT surface. FIG. 8A shows the azurin attachment scheme. Samples containing irradiated CNT bundles were immersed in a 5 ml bath of deionized water containing 1 mg azurin (maintained at a pH ˜5) for 2 hours. The pH was maintained below the pI of azurin to retain a residual positive charge on the biomolecule to enable attachment. The sample was then removed, washed thoroughly with deionized water and dried prior to characterization by Raman spectroscopy. Selected samples of azurin were also marked with Au nanoparticles prior to attachment with irradiated CNTs, to enable visualization by SEM. FIGS. 8B and 8C show attachment of azurin on irradiated CNTs but not on non-irradiated CNTs, respectively, thus confirming site-selective attachment. Attachment occurred only for pH<8, which corresponds to the pI of azurin, indicating electrostatic anchoring. Irrespective of the pH, there was no detectable attachment of the azurin on to unirradiated CNTs. FIG. 8D shows spatially resolved micro-Raman spectra that confirm azurin's attachment only onto irradiated CNT segments. Azurin-anchored CNT segments exhibit two strong Raman modes at 291 and 353 cm⁻¹ that are characteristics of Cu—S (Cys) stretching in tetragonal or distorted tetrahedral coordination. These spectral signatures are not detectable in non-irradiated CNTs treated with azurin, confirming site-selective anchoring of the protein to irradiated CNTs. Spectra from pristine azurin also show Cu—S (Cys) stretching modes, but at higher wave numbers of 377 and 414 cm⁻¹ (with a shoulder at 435 cm⁻¹) associated with trigonal planar coordination. Thus, azurin's structure, and hence its polarization, are altered upon immobilization on a CNT. However, the retention of the Cu—S (Cys) spectral signatures indicates that that the immobilized protein is quite robust in nonphysiological environments and retains it redox-activity. These features are promising for realizing protein-CNT devices where the correlation between the protein conduction state and the type of immobilization is used as means to fingerprint and detect analytes, or tune protein orientation and activity with analytes via electrical signals through the CNT.

The foregoing description of the invention has been presented for purposes of illustration and description. It is not intended to be exhaustive or to limit the invention to the precise form disclosed, and modifications and variations are possible in light of the above teachings or may be acquired from practice of the invention. The description was chosen in order to explain the principles of the invention and its practical application. It is intended that the scope of the invention be defined by the claims appended hereto, and their equivalents.

1. A carbon nanotube comprising:
at least one functional group in a site-selective functionalization on a surface of the nanotube; or
a plurality of functional groups in an ordered arrangement on a surface of the nanotube.

2. The carbon nanotube of claim 1, comprising the at least one functional group in the site-selective functionalization on the surface of the nanotube.

3. The carbon nanotube of claim 2, wherein the at least one functional group is bound to an ion irradiated portion of the surface of the nanotube and not to a non-irradiated portion of the surface of the nanotube.

4. The carbon nanotube of claim 2, wherein:
the site-selective functionalization comprises a binding of the at least one functional group to an ion irradiated induced defect site on the surface of the nanotube; and
the binding comprises at least one of a covalent binding or a Van der Waals binding.

5. The carbon nanotube of claim 1, comprising the plurality of functional groups in the ordered arrangement on the surface of the nanotube.

6. The carbon nanotube of claim 5, wherein the ordered arrangement comprises at least one first portion of the surface of the nanotube containing a higher concentration of functional groups than at least one second portion of the nanotube surface.

7. The carbon nanotube of claim 6, wherein the at least one first portion comprises an irradiated portion of the surface of the nanotube.

8. The carbon nanotube of claim 1, comprising:
a plurality of functional groups in the site-selective functionalization on the surface of the nanotube; and
the plurality of functional groups in the ordered arrangement on the surface of the nanotube.

9. The carbon nanotube of claim 8, wherein the functional groups are selected from the group consisting of:
an alcohol;
a carbonyl;
a carboxyl; or
an allyl.

10. The carbon nanotube of claim 8, further comprising a nanostructure that is selectively attached to the functional group, wherein the nanostructure is selected from the group consisting of:
a nanoparticle;
a nanosphere;
an amino acid; or
a protein.

11. The carbon nanotube of claim 10, wherein:
(a) the functional groups comprise a carboxyl and the nanostructure comprises the nanoparticle;
(b) the functional groups comprise an allyl and the nanostructure comprises the nanosphere;
(c) the functional groups comprise a carboxyl and the nanostructure comprises the amino acid; or
(d) the functional groups comprise a carboxyl and the nanostructure comprises the protein.

12. The carbon nanotube of claim 11, wherein:
(a) the nanoparticle comprises a negatively-charged gold nanoparticle and a cationic polyelectrolyte is located between the nanoparticle and the functional groups;
(b) the nanosphere comprises a carboxylated nanosphere and the allyl comprises a brominated allyl whose bromine atom is displaced by the carboxylated nanosphere;
(c) the amino acid comprises lysine; or
(d) the protein comprises azurin.

13. A device comprising the nanotube of claim 10, wherein the device is adapted to detect or utilize a selective attachment of the nanostructure to the device.

14. A method of functionalizing a carbon nanotube, comprising:
providing a carbon nanotube;
providing ions at a dose greater than 10¹³ ions cm⁻² having an energy greater than 1 keV on at least one first portion of the nanotube surface; and
exposing the nanotube to an oxygen-containing medium such that at least one functional group is formed on the at least one first portion of the nanotube surface.

15. The method of claim 14, wherein:
the ions are Ga⁺ or Ar⁺ ions;
the dose is 10¹⁵-10¹⁷ ions cm⁻²; and
the energy is 5-30 keV.

16. The method of claim 15, wherein:
the ions are provided by focused ion beam irradiation; and
the oxygen-containing medium is air or water.

17. The method of claim 14, further comprising:
selectively attaching a nanostructure to the at least one functional group.

18. The method of claim 17, wherein the step of selectively attaching comprises at least one of:
(a) providing a nanostructure comprising a negatively-charged gold nanoparticle and a cationic polyelectrolyte, wherein the at least one functional group comprises a carboxyl;
(b) providing a nanostructure comprising a carboxylated microsphere, wherein the at least one functional group comprises an allyl;
(c) providing a nanostructure comprising an amino acid, wherein the at least one functional group comprises a carboxyl; or
(d) providing a nanostructure comprising a protein, wherein the at least one functional group comprises a carboxyl.

19. A method of functionalizing a carbon nanotube, comprising:
providing a carbon nanotube;
irradiating at least one exposed portion of the nanotube surface with ions to generate at least one defect site on the at least one exposed portion; and
forming at least one functional group on the at least one defect site.

20. The method of claim 19, wherein the step of irradiating comprises selectively irradiating a predetermined area of the nanotube surface.

21. The method of claim 19, further comprising attaching a nanostructure only to an irradiated portion of the nanotube surface and not to non-irradiated portions of the nanotube surface.

22. The method of claim 21, wherein:
the at least one functional group is selected from the group consisting of:
an alcohol;
a carbonyl;
a carboxyl; and
an allyl; and
the nanostructure is selected from the group consisting of:
a nanoparticle;
a nanosphere;
an amino acid; and
a protein.
